(19) United States
(12) Patent Application Publication
(10) Pub. No.: US 2017/0319866 A1
(43) Pub. Date: Nov. 9, 2017

(54) MULTI-FUNCTIONAL AIRBAG

(71) Applicant: Guangdong Travelmall Health Technology Co., Ltd., Dongguan (CN)

(72) Inventor: Zhiming Liu, Dongguan (CN)

(21) Appl. No.: 15/638,762

(22) Filed: Jun. 30, 2017

(30) Foreign Application Priority Data
Jul. 16, 2016 (CN) 201610558504

(51) Int. Cl. A61N 2/00; A61F 7/00; A61H 23/02; A61N 5/06; A61H 39/00

(52) U.S. Cl. CPC A61N 2/002; A61N 5/0619; A61F 7/007; A61H 23/0254; A61H 39/007; A61H 2201/5007; A61H 2201/1609; A61H 2201/1215; A61H 2201/0207; A61H 2201/0103; A61F 2007/0096; A61F 2007/0011; A61H 2201/5071; A61H 2205/02; A61H 2205/084; A61N 2005/0659; A61N 2005/0645

(57) ABSTRACT

A multi-functional airbag comprises a main body, a front side of the main body is provided with an opening, a rear side of the main body opposite to the opening is provided with a functional cavity to install a functional assembly, a left air chamber and a right air chamber communicated with each other are symmetrically configured at two sides of the main body, and the functional assembly is installed in the functional cavity. While in use, the air chambers are inflated, as a result the main body is inflated to enable to clamp two sides of the human neck; while not in use, the air chambers are deflated to reduce the volume to make it portable. Meanwhile, massage functioning members are configured to massage the acupuncture points by vibration therapy, pulse therapy, heat therapy, infrared light therapy, and/or magnet therapy, and the therapy effect is obvious.

### RELATED APPLICATIONS

This application claims the benefit of priority to Chinese Invention Application No. 201610558504.4, filed Jul. 16, 2016, which is hereby incorporated by reference in its entirety.

### FIELD OF THE INVENTION

The invention relates to a technical field of human health products, and more particularly to a multi-functional airbag.

### BACKGROUND OF THE INVENTION

Current massage devices or massagers mainly includes a massage assembly constituting of a motor and a plastic member, a plastic frame or a foamed sponge supporting the massage assembly, a power supplier, a control switch and a case. This massager has a bulky size and the plastic case is hard, which is inconvenient to carry and use, thus the massager is only applicable to fixed place such as home, offices, stations or health experience stores and the like. Unfortunately, it's helpless for some peoples such as on-trip business man, outdoor tourists, office workers, and soldiers etc. who stay outside for long term to use these massage devices. As the people's aging problems become more and more significant, a number of these people got multiple occupational diseases such as cervical spondylosis, frozen shoulder, lumbar disease, eye fatigue etc.

### SUMMARY OF THE INVENTION

To overcome the above-mentioned drawbacks, a multi-functional airbag that is portable and has obvious therapy effect is provided.

The present invention is achieved by the following technical solutions.

A multi-functional airbag comprises a main body, a front side of the main body provided with an opening to allow a neck to pass through, a rear side of the main body opposite to the opening provided with a functional cavity to install a functional assembly, wherein the functional cavity includes a functional cavity front wall for contacting with the neck and a functional cavity rear wall opposite to the functional cavity front wall, a functional cavity opening is provided at the functional cavity rear wall and communicated with the functional cavity, a left air chamber and a right air chamber communicated with each other are symmetrically configured at two sides of the main body, the left air chamber and the right air chamber are connected with an inflation device which is selected from a press-type inflation device, an electric inflation device and an air nozzle, and the functional cavity is equipped with the functional assembly arranged for human health and physiotherapy of human body.

As an improvement of the present invention, the functional assembly comprises a mounting plate, at least one functioning member mounted on the mounting plate to contact with human body acupuncture points, a battery for supplying power for the functioning member, a control circuit board for controlling the functioning member, and a control panel, wherein a front end of the functioning member is protruded from a through hole formed on the functional cavity front wall to contact with the human body acupuncture points on the neck, and the control panel is configured at the opening of the functional cavity.

As an improvement of the present invention, a heating area is configured at an outer side of the functional cavity front wall, and a heating element is configured in a corresponding inner side of the functional cavity front wall, a thermistor is included to detect temperature of the heating area, and the heating element and the thermistor are electrically connected with the control circuit board.

As an improvement of the present invention, the main body is circular, and each end of the main body adjacent to the opening are provided with an end cover respectively.

As an improvement of the present invention, the electric inflation device is located in the functional cavity and mounted on the mounting plate, the electric inflation device includes a mini inflatable pump communicated with the left air chamber and the right air chamber respectively, and a pressure sensor is included to detect pressure in the left air chamber and the right air chamber, both the mini inflatable pump and the pressure sensor are electrically connected with the control circuit board.

As an improvement of the present invention, the amount of the functioning member is three, and the functioning members are aligned with left Tianchi point, right Tianchi point and Tianzhu point respectively.

As an improvement of the present invention, the functioning member is acted on the human body acupuncture points with at least one selected from pulse action effect, vibration action effect, infrared light action effect, heat action effect, and magnet action effect.

As an improvement of the present invention, the functioning member includes an functioning member case which is provided with at least one selected from a magnet, an infrared generator and a vibration motor, and the functioning member is selected from silicone, rubber and PVC material.

As an improvement of the present invention, the functioning member case is a conductive silicone case which is provided with an acting surface contacting with the human body acupuncture points, a magnet is mounted in an inner side of the conductive silicone case, a vibration motor is fixedly connected with the conductive silicone case and electrically connected with the control circuit board, and the conductive silicone case is electrically connected the control circuit board to receive a pulse current.

As an improvement of the present invention, the magnet is replaced by the infrared generator, and a window for emitting infrared lights is formed on the acting surface of the conductive silicone case and electrically connected with the control circuit board.

A multi-functional airbag comprises a main body, a front side of the main body provided with an opening to allow a neck to pass through, a rear side of the main body opposite to the opening provided with a functional cavity to install a functional assembly, wherein the functional cavity includes a functional cavity front wall for contacting with the neck and a functional cavity rear wall opposite to the functional cavity front wall, a left air chamber and a right air chamber are configured at two sides of the main body, both of the left air chamber and the right air chamber are hermetically separated with the functional cavity, the left air chamber and the right air chamber are connected with at least one inflation device which is selected from a press-type inflation device, an electric inflation device and an air nozzle, and the functional cavity is equipped with the functional assembly arranged for human health and physiotherapy of human body.

In comparison with the prior art, as the main body of the present invention is made of silicone material, which includes a left air chamber and a right air chamber that are inflatable. While in use, the left and right air chambers are inflated by the inflation device, as a result the main body is inflated to enable to clamp two sides of the human neck; while not in use, the left and right air chambers are deflated to reduce the volume of the device, so as to make it portable. Meanwhile, massage functioning members are configured to massage the acupuncture points by vibration therapy, pulse therapy, heat therapy, infrared light therapy, and/or magnet therapy, and the therapy effect is obvious.

### BRIEF DESCRIPTION OF THE DRAWINGS

The accompanying drawings facilitate an understanding of the various embodiments of this invention. In such drawings:

FIG. 1 is a perspective view of a multi-functional airbag according to an embodiment of the present invention;

FIG. 2 is a structure view of the main body according to the present invention;

FIG. 3 is a structure view of the functional assembly according to the present invention;

FIG. 4 is an exploded view of the multi-functional airbag according to the present invention;

FIG. 5 is a structure view of a functioning member according to a first embodiment of the present invention;

FIG. 6 is a structure view of a functioning member according to a second embodiment of the present invention;

FIG. 7 is a schematic view of the multi-functional according to the first embodiment of the present invention;

FIG. 8 is a perspective view of a multi-functional airbag according to another embodiment of the present invention;

FIG. 9 is another perspective view of the multi-functional airbag of FIG. 8;

FIG. 10 is a perspective view of a multi-functional airbag according to one more embodiment of the present invention, with a portion of the main body is removed to expose the air chamber;

FIG. 11 is a perspective view of a multi-functional airbag according to one more embodiment of the present invention;

FIG. 12 is an exploded view of FIG. 11; and

FIG. 13 is a partial view of the multi-functional airbag, with a portion of the main body is removed to expose the air chamber.

### DETAILED DESCRIPTION OF ILLUSTRATED EMBODIMENTS

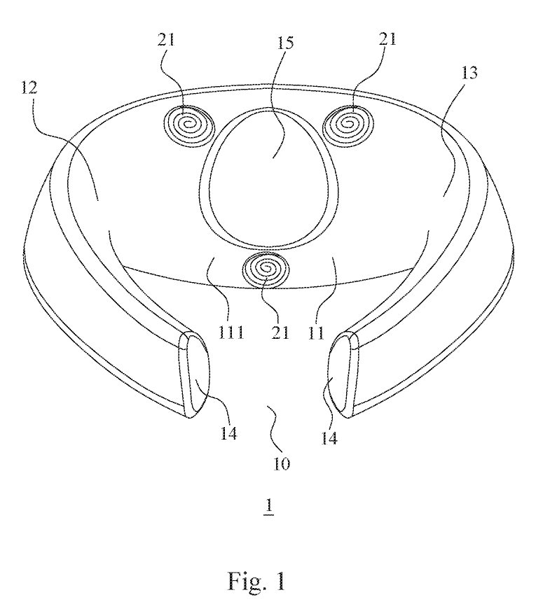

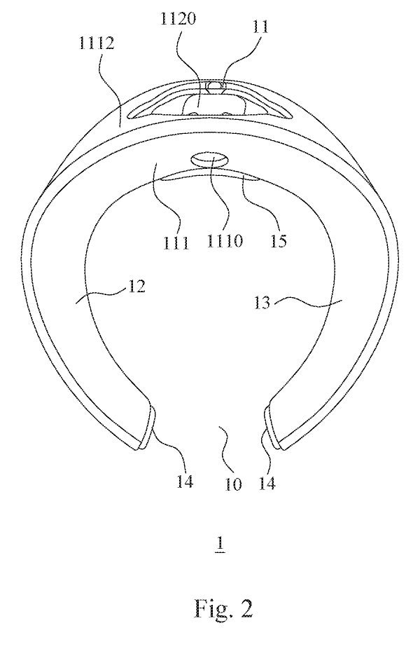

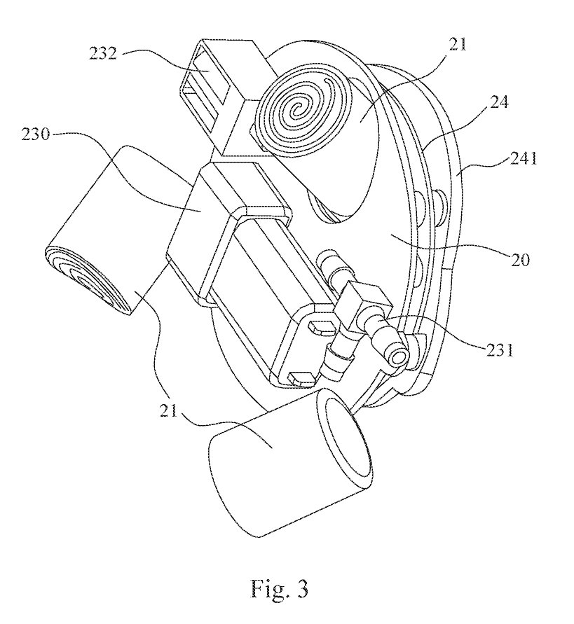

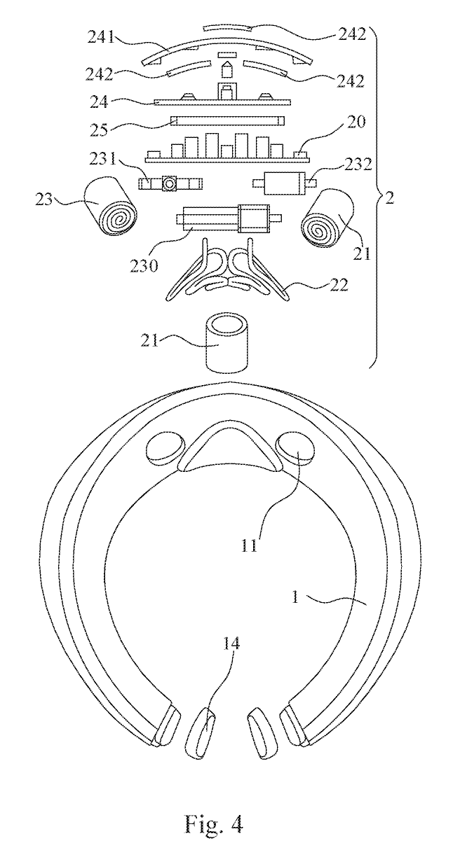

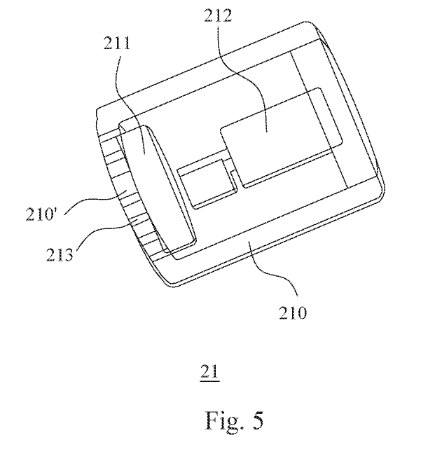

Referring to FIGS. 1 to 5, a multi-functional airbag according to a first embodiment of the present invention includes a main body 1 which is made of silicone material, a front side of the main body 1 is provided with an opening 10 to allow a human neck to pass through, a rear side of the main body 1 opposite to the opening 10 is provided with a functional cavity to install a functional assembly 2. The functional cavity 11 includes a functional cavity front wall 111 for contacting with the neck and a functional cavity rear wall 112 opposite to the functional cavity front wall 111, a functional cavity opening 1120 is provided on the functional cavity rear wall 112 and communicated with the functional cavity 11, a left air chamber 12 and a right air chamber 13 communicated with each other are symmetrically configured at two sides of the main body 1, the left air chamber 12 and the right air chamber 13 are connected with an inflation device which is at least one selected from a press-type inflation device, an electric inflation device and an air nozzle. Preferably, the electric inflation device is used in this embodiment. Alternatively, the left air chamber 12 and a right air chamber 13 may be configured asymmetrically.

The functional assembly 2 is installed in the functional cavity 11, which includes a mounting plate 20, three functioning member 21 mounted on the mounting plate 20, a rechargeable lithium battery 25 for supplying power for the functioning member 21, a control circuit board 24 and a control panel 241. Specifically, a front end of the functioning member 21 is protruded from three through holes 1110 formed on the functional cavity front wall 111 to contact with the acupuncture points on the neck. A raised heating area 15 is configured at the outer side of the functional cavity front wall 111 and among the functioning members 21, a heating element 22 is configured in a corresponding inner side of the functional cavity front wall 111 and electrically connected with the control circuit board 24. The control panel 241 is configured at the functional cavity opening 1120. In this embodiment, the heating element 15 applies heating coils, and the electric inflation device is configured on the mounting plate 20 as well.

Specifically, the main body 1 is made of silicone material and formed in a unity structure. The thickness of the side wall of the functional cavity 11 is larger than that of the side walls of the left and right air chambers 12, 13, as a result, the thicker functional cavity 11 can protect and fix the thinner left and right air chambers 12, which is beneficial to inflate or deflate. While in use, the left and right air chambers 12, 13 are inflated to enable to clamp at two sides of the neck, thereby fixing the device stably, while not in use, the left and right air chambers 12, 13 are deflated to reduce size thereby enabling to be portable and folded.

The left air chamber 12 and the right air chamber 13 are communicated with each other by an air tube connector 231 in the functional cavity 11 and communicated with the electric inflation device by this air tube connector 231. Two ends of the main body at the front side adjacent to the opening 10 are provided with an end cover 14 respectively for decoration.

The electric inflation device includes a mini inflatable pump 230 which is communicated with the left air chamber 12 and the right air chamber 13 via an electromagnetic valve 232 and an air tube connector 231. Further, a pressure sensor (not shown) is included to detect the pressure in the left and right air chambers 12 and 13. Specifically, the mini inflatable pump 230, the pressure sensor and the electromagnetic valve 232 are electrically connected with the control circuit board 24. While in use, the pressure sensor will firstly detect the pressure in the air chambers, the mini inflatable pump 230 will be controlled by the control circuit board 24 to inflate the air chambers if a predetermined working pressure value is not achieved; otherwise, the inflation to the air chambers will be stopped to maintain the constant pressure if the predetermined working pressure is achieved. While not in use, the electromagnetic valve 232 will cause the air chambers to communicate with the outside to deflate, thereby reducing the volume of the airbag to make it portable.

The functioning members are aligned with left Tianchi point, right Tianchi point and Tianzhu point respectively. Specifically, the three functioning member are distributed at three vertexes of a triangle, each of which includes a conductive silicone case 210. More specifically, the conductive silicone case 210 is in a cylindrical shape and includes a hollow cavity, and the conductive silicone case 210 includes an acting surface 210′ contacting with the human body acupuncture points and a side wall surrounding the acting surface 210′. Further, an infrared generator 211 is installed in the inner side of the acting surface 210′, and a window 213 for emitting infrared lights is formed on the acting surface 210′. A vibration motor 212 is fixedly connected to the conductive silicone case 210 and electrically connected with the control circuit board 24, preferably, this vibration motor 212 is an ordinary motor with an eccentric weight connected on the rotating axis. The back end of the conductive silicone case 210 is electrically connected with the control circuit board 24 to receive a pulse current, and will be vibrated along with the vibration motor 212 when the motor 212 is working.

Preferably, the conductive silicone case 210 is made of transparent silicone material, whose surface except for the window 213 is coated with a colored ink. In such a way, when the infrared generator 211 is working, infrared light is emitted from the window 213 to irradiate on the acupuncture points of the neck, so as to generate heat effect of infrared light on the neck.

Multiple control keys 242 and LED indicators are formed on the control panel 241, and the LED indicators electrically connected with the control circuit board 24 are used for indicating the battery capacity. Furthermore, the control panel 241 is provided with an interface by which the rechargeable lithium battery 25 can be charged.

Moreover, a thermistor (not shown) is included to detect the temperature of the heating area 15, and the thermistor is electrically connected with the control circuit board 24, so that the temperature of the heating area 15 can be set; as a result, the control circuit board 24 can control to decrease the temperature if the heating area 15 is overheated, otherwise, control to increase the temperature if a predetermined temperature is not achieved, and then maintain the constant temperature.

In such a way, when the functioning members 21 contact with the human body acupuncture points to carry out vibration massage therapy, the conductive silicone case 210 can generate a pulse current to stimulate the acupuncture points to carry out pulse therapy. In addition, due to the infrared light irradiation from the infrared generator 211, heat effect can be produced to the acupuncture points to carry out infrared light therapy. Meanwhile, the heating area 15 is pressed against to the neck zone among the three acupuncture points to carry out heat therapy on the rear neck.

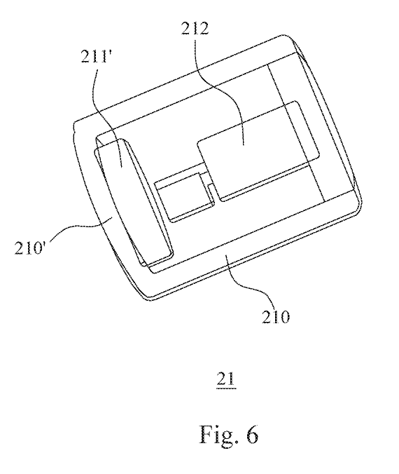

A multi-functional airbag according to a second embodiment has the similar structure as the first embodiment, differences are that, the infrared generator 211 in the conductive silicone case 210 is replaced by a magnet 211′, and the window 213 on the acting surface 210′ is omitted, as shown in FIG. 6. Due to the magnet 211′, the functioning member 21 has the effect of magnet therapy.

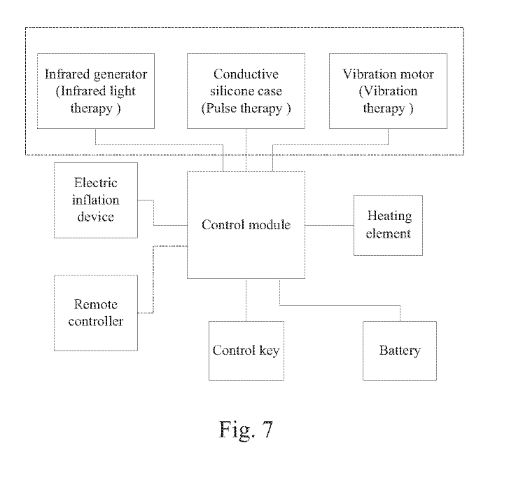

As shown in FIG. 7, the control module is configured on the control circuit board which is a Single Chip Microcomputer (SCM) generally, and a remote controller is also included to remote control the present device by infrared light connection or Bluetooth connection.

The control module can be preset with multiple working mode, such as:

Mode 1: heat therapy;

Mode 2: pulse therapy;

Mode 3: vibration massage therapy;

Mode 4: heat and pulse therapy; and

Mode 5: head and vibration massage therapy.

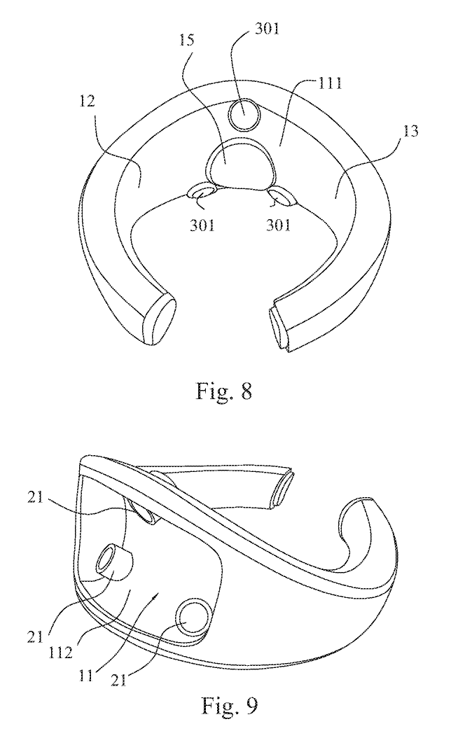

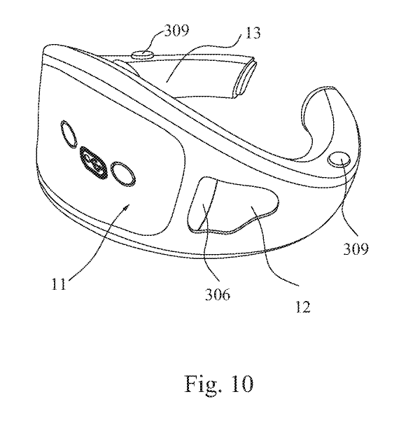

FIG. 8 is a perspective view of a multi-functional airbag according to another embodiment of the present invention; FIG. 9 is another perspective view of the multi-functional airbag of FIG. 8. The structure of the multi-functional airbag in this embodiment is similar to that of the first one, but differences are that, the front end of the functioning member 21 is not protruded from the through hole of the functional cavity front wall 111, instead, the functional cavity front wall 111 has at least one raised receiving portion 301 for accommodating the functioning member 21. Specifically, the functioning member 21 inside the functional cavity is pressed against an inner surface of the raised receiving portion 301 to stimulate the acupuncture point.

Preferably, the inflation device in this embodiment is an electric inflation device (not shown). The amount of the electric inflation device can be one or two, for example, a single electric inflation device is arranged in the functional cavity 11 and communicated with the left and right air chambers 12 and 13 respectively; or, two individual electric inflation devices are arranged to connect with the left and right air chambers 12 and 13 respectively.

Preferably, as shown in FIG. 10, two inflation devices 309 are arranged in the left air chamber 12 and the right air chamber 13 respectively, in this case, the left air chamber 12 and the right air chamber 13 are not communicated with each other. Specifically, the left air chamber 12 and the right air chamber 13 are individual and hermetically separated with the functional cavity 11, as shown in FIG. 10, a wall 306 is arranged between the left air chamber 12 and the functional cavity 11 to achieve the hermetical separation; Similarly, a wall (not shown) is arranged between the right air chamber 13 and the functional cavity 11 to achieve the hermetical separation. As an example, the inflation device 309 can be a press-type inflation device 309.

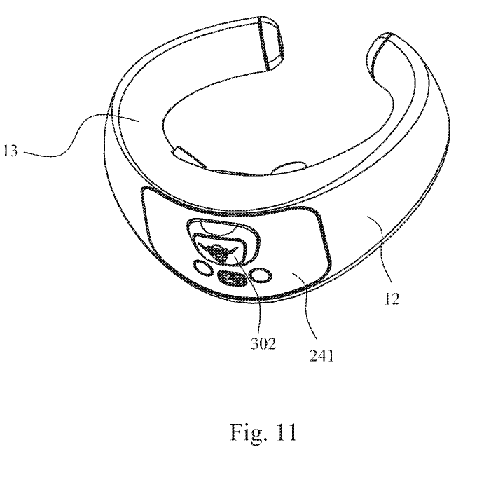

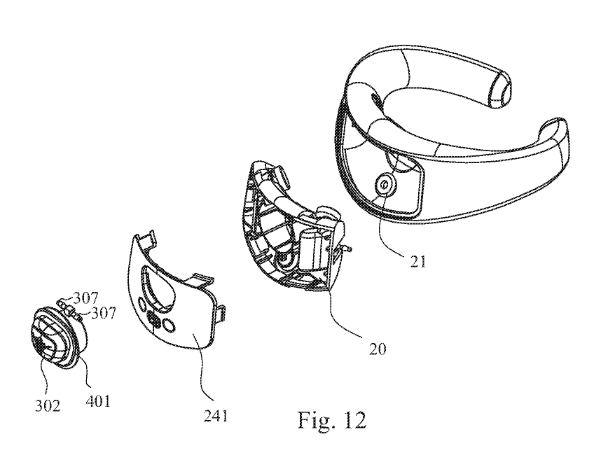

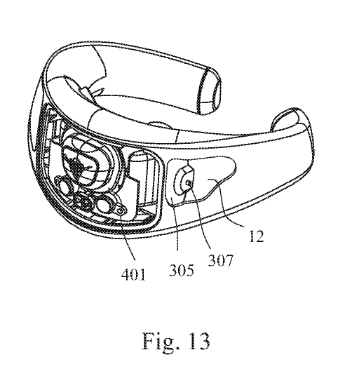

FIG. 11 is a perspective view of a multi-functional airbag according to one more embodiment of the present invention; FIG. 12 is an exploded view of FIG. 11; and FIG. 13 is a partial view of the multi-functional airbag. In this embodiment, a single press-type inflation device 401 is used. Specifically, the press-type inflation device 401 is arranged in the functional cavity 11 and connected with the left and right air chambers 12 and 13 by air tubes. The press-type inflation device has a press area 302 protruding from the control panel 241. By pressing the press area 302, the left and right air chambers 12 and 13 can be inflated or deflated. Specifically, as shown in FIG. 12, the press-type inflation device 401 is mounted on the mounting plate 20, two air tube connectors 307 are connected with the press-type inflation device 401 and extended to the left and right air chamber 12 and 13 respectively, as shown in FIG. 13. Specifically, a wall 305 between the left air chamber 12 and the functional cavity 11 is provided with a hole to allow the air tube connector 307 to pass through. Similarly, a wall (not shown) between the right air chamber 13 and the functional cavity 11 is also provided with a hole to allow the air tube connector 307 to pass through. In such a way, the left and right air chamber 12, 13 are communicated with each other, and hermetically separated with the functional cavity 11.

While the invention has been described in connection with what are presently considered to be the most practical and preferred embodiments, it is to be understood that the invention is not to be limited to the disclosed embodiments, but on the contrary, is intended to cover various modifications and equivalent arrangements included within the spirit and scope of the invention.

What is claimed is:

1. A multi-functional airbag, comprising a main body, a front side of the main body provided with an opening to allow a neck to pass through, a rear side of the main body opposite to the opening provided with a functional cavity to install a functional assembly, wherein the functional cavity includes a functional cavity front wall for contacting with the neck and a functional cavity rear wall opposite to the functional cavity front wall, a functional cavity opening is provided at the functional cavity rear wall and communicated with the functional cavity, a left air chamber and a right air chamber communicated with each other are symmetrically configured at two sides of the main body, both of the left air chamber and the right air chamber are hermetically separated with the functional cavity, the left air chamber and the right air chamber are connected with an inflation device which is selected from a press-type inflation device, an electric inflation device and an air nozzle, and the functional cavity is equipped with the functional assembly arranged for human health and physiotherapy of human body.

2. The multi-functional airbag according to claim 1, wherein the functional assembly comprises a mounting plate, at least one functioning member mounted on the mounting plate to contact with human body acupuncture points, a battery for supplying power for the functioning member, a control circuit board for controlling the functioning member, and a control panel, wherein a front end of the functioning member is protruded from a through hole formed on the functional cavity front wall to contact with the human body acupuncture points on the neck, and the control panel is configured at the opening of the functional cavity.

3. The multi-functional airbag according to claim 1, wherein a heating area is configured at an outer side of the functional cavity front wall, and a heating element is configured in a corresponding inner side of the functional cavity front wall, a thermistor is included to detect temperature of the heating area, and the heating element and the thermistor are electrically connected with the control circuit board.

4. The multi-functional airbag according to claim 1, wherein the main body is circular, and each end of the main body adjacent to the opening are provided with an end cover respectively.

5. The multi-functional airbag according to claim 2, wherein the electric inflation device is located in the functional cavity and mounted on the mounting plate, the electric inflation device includes a mini inflatable pump communicated with the left air chamber and the right air chamber respectively, and a pressure sensor is included to detect pressure in the left air chamber and the right air chamber, both the mini inflatable pump and the pressure sensor are electrically connected with the control circuit board.

6. The multi-functional airbag according to claim 2, wherein the amount of the functioning member is three, and the functioning members are aligned with left Tianchi point, right Tianchi point and Tianzhu point respectively.

7. The multi-functional airbag according to claim 2, wherein the functioning member is acted on the human body acupuncture points with at least one selected from pulse action effect, vibration action effect, infrared light action effect, heat action effect, and magnet action effect.

8. The multi-functional airbag according to claim 2, wherein the functioning member includes an functioning member case which is provided with at least one selected from a magnet, an infrared generator and a vibration motor, and the functioning member is selected from silicone, rubber and PVC material.

9. The multi-functional airbag according to claim 8, wherein the functioning member case is a conductive silicone case which is provided with an acting surface contacting with the human body acupuncture points, a magnet is mounted in an inner side of the conductive silicone case, a vibration motor is fixedly connected with the conductive silicone case and electrically connected with the control circuit board, and the conductive silicone case is electrically connected the control circuit board to receive a pulse current.

10. The multi-functional airbag according to claim 9, wherein the magnet is replaced by the infrared generator, and a window for emitting infrared lights is formed on the acting surface of the conductive silicone case and electrically connected with the control circuit board.

11. A multi-functional airbag, comprising a main body, a front side of the main body provided with an opening to allow a neck to pass through, a rear side of the main body opposite to the opening provided with a functional cavity to install a functional assembly, wherein the functional cavity includes a functional cavity front wall for contacting with the neck and a functional cavity rear wall opposite to the functional cavity front wall, a left air chamber and a right air chamber are configured at two sides of the main body, both of the left air chamber and the right air chamber are hermetically separated with the functional cavity, the left air chamber and the right air chamber are connected with at least one inflation device which is selected from a press-type inflation device, an electric inflation device and an air nozzle, and the functional cavity is equipped with the functional assembly arranged for human health and physiotherapy of human body.

12. The multi-functional airbag according to claim 11, wherein the left air chamber is connected with a first inflation device, and the right air chamber is connected with a second inflation device.

13. The multi-functional airbag according to claim 11, wherein the functional assembly comprises a mounting plate, at least one functioning member mounted on the mounting plate to contact with human body acupuncture points, a battery for supplying power for the functioning member, a control circuit board for controlling the functioning member, and a control panel; a functional cavity opening is provided at the functional cavity rear wall and communicated with the functional cavity, and the control panel is configured at the opening of the functional cavity.

14. The multi-functional airbag according to claim 13, wherein the functional cavity front wall has at least one raised receiving portion for accommodating the functioning member.

15. The multi-functional airbag according to claim 11, wherein a heating area is configured at an outer side of the functional cavity front wall, and a heating element is configured in a corresponding inner side of the functional cavity front wall, a thermistor is included to detect temperature of the heating area, and the heating element and the thermistor are electrically connected with the control circuit board.

16. The multi-functional airbag according to claim 13, wherein the amount of the functioning member is three, and the functioning members are aligned with left Tianchi point, right Tianchi point and Tianzhu point respectively.

17. The multi-functional airbag according to claim 13, wherein the functioning member is acted on the human body acupuncture points with at least one selected from pulse action effect, vibration action effect, infrared light action effect, heat action effect, and magnet action effect.

18. The multi-functional airbag according to claim 13, wherein the functioning member includes an functioning member case which is provided with at least one selected from a magnet, an infrared generator and a vibration motor, and the functioning member is selected from silicone, rubber and PVC material.

19. The multi-functional airbag according to claim 18, wherein the functioning member case is a conductive silicone case which is provided with an acting surface contacting with the human body acupuncture points, a magnet is mounted in an inner side of the conductive silicone case, a vibration motor is fixedly connected with the conductive silicone case and electrically connected with the control circuit board, and the conductive silicone case is electrically connected the control circuit board to receive a pulse current.

20. The multi-functional airbag according to claim 19, wherein the magnet is replaced by the infrared generator, and a window for emitting infrared lights is formed on the acting surface of the conductive silicone case and electrically connected with the control circuit board.
